(19) United States
(12) Patent Application Publication
(10) Pub. No.: US 2013/0197334 A1
(43) Pub. Date: Aug. 1, 2013

(54) BIODETECTOR

(71) Applicants: Ekkehardt Weber, Halle (Saale) (DE); Robert Niestroj, Berlin (DE)

(72) Inventors: Ekkehardt Weber, Halle (Saale) (DE); Robert Niestroj, Berlin (DE)

(73) Assignee: GILUPI GMBH, POTSDAM OT GO IM (DE)

(21) Appl. No.: 13/635,568

(22) PCT Filed: Mar. 15, 2011
(86) PCT No.: PCT/EP11/01293

§ 371 Date: Oct. 23, 2012

(30) Foreign Application Priority Data
Mar. 16, 2010 (DE) 10 2010 011 560.6

(51) Int. Cl. A61B 5/15; A61B 5/157; A61B 5/145; A61B 5/153

(52) U.S. Cl. CPC A61B 5/150358; A61B 5/153; A61B 5/157; A61B 5/14546; A61B 5/150389
USPC 600/362

(57) ABSTRACT

The invention concerns a Biodetector with a functionalised surface for isolating molecules or cells from the human body. In order to improve a Biodetector of the type mentioned above such that the functionalised surface of the Biodetector or there upon enriched molecules or cells are exposed to a lower abrasion, and the biocompatibility of the Biodetector is improved, the Biodetector is designed to remove a fluid from the human body and absorb it into an inner space of the Biodetector, wherein the functionalised surface is oriented to the inner space of the Biodetector.

The invention concerns a biodetector with a functionalised surface for isolating molecules or cells from the human body.

Such a biodetector is known for example from WO 2006/131400 A1. This Biodetector is introduced into the human body for the isolation and enrichment of target molecules and target cells and after a short period is once again removed from the human body. When introducing the biodetector into the body or removing it the functionalised surface or there upon enriched material is abraded easily. It should be ensured as much as possible that the functionalised surface of biodetector is biocompatible and does not cause immune reactions.

The underlying purpose of the invention is to improve the Biodetector of the type mentioned above such that the functionalised surface of the biodetector or there upon enriched molecules or cells are exposed to a lower abrasion, and the biocompatibility of the biodetector is improved.

To solve the underlying problem of the invention, the invention provides the biodetector with a functionalised surface for the isolation of molecules or cells from the human body according to claim 1, which is designed in order to remove fluid from the human body, and to absorb it into an inner space of Biodetector wherein the functionalised surface is oriented to the biodetector. By using a Biodetector according to the invention, a certain amount of body fluid can be removed and absorbed into the Biodetector and analysed if necessary. The functionalised surface and the there upon enriched target molecules or target cells are protected from abrasion in the interior of the Biodetector. Further, body fluid which comes into contact with the functionalised surface is not improperly recycled back into the body and is no longer part of the circulating fluid amount in the body. Thus, the use of human components on the functionalised surface is no longer necessary and all the biocompatibility tests can be omitted.

Preferred further improvements of the invention are the subjects of additional/dependent claims

It may prove to be advantageous if the functionalised biodetector is at least partially occupied with detection molecules or a receptor or ligand. This allows selected target molecules and target cells to be easily enriched.

It would be further deemed advantageous if as detection molecules antibodies, preferably monoclonal antibodies, chimeric antibodies, humanized antibodies, antibody fragments or amino acid structures and amino acid sequences, nucleic acid structures or nucleic acid sequences, or carbohydrate structures or synthetic structures are used.

But it may also be useful if the detection molecules are of non-human origin, preferably of animal origin, or murine origin. Such detection molecules allow a wider range for the enrichment of target molecules and target cells than human detection molecules alone.

It may prove to be helpful if the detection molecules, preferably by means of Protein G, are attached orienting to the functionalised surface. Protein G exhibits a high affinity for specific immunoglobulins.

But it is also considered practical when the detection molecules are covalently attached to the functionalised surface.

In an advantageous improvement of the invention, the Biodetector features a fluid removal section that meets at least one of the following requirements:
The fluid removal section includes an opening through which the fluid enters the inner space of the biodetector. This allows the absorption of the fluid to be properly controlled.
An inner space of fluid removal section communicates with the opening. Thereby the fluid removal section can already absorb a certain amount of fluid by itself.
The opening is formed at one end of the fluid removal section. Thereby the amount of fluid absorbed is conducted only in one direction through the fluid removal section. The risk of an uncontrolled back flow of the absorbed fluid can be reduced substantially.
The fluid removal section is at least partially made of a biocompatible material. Thus, an immune response of the human body is largely prevented.
The fluid removal section is at least partially made of plastic, metal or glass, preferably stainless steel, titanium, or fibre, more preferably of a biocompatible plastic or a combination of these substances. Such materials are easily moulded.
The fluid removal section is designed in the form of a cannula. Cannulas are standardized and available in various sizes. Through the use of standardized components, the manufacturing costs of the biodetector are reduced.
The fluid removal section comprises an outer diameter of 0.25 to 3.5 mm, preferably 0.5 to 3.0 mm, more preferably 0.75 to 2.5 mm, and even more preferably 1.0 mm to 2.0 mm. Such cannula sizes are particularly common.
The opening comprises a diameter of 0.2 to 3.0 mm, preferably 0.25 to 2.5 mm, more preferably 0.5 to 2.0 mm, even more preferably 0.75 mm to 1.5 mm. Through an aperture of this size the amount of fluid absorbed can be properly controlled.
The functionalised surface is located on an inner wall of the fluid removal section. Thereby; the interior of the fluid removal section can be already used for the enrichment of the target molecules or the target cells. As a result, the amount of the fluid to be removed from the human body used for the isolation of molecules or cells can be reduced.
The fluid removal section at least partially consists of a material which contains functional groups for covalent binding of the detection molecule, and/or which contains chemically or enzymatically fissile groups in order to facilitate the quantitative recovery of bound target molecules, or target cells, and/or forms a matrix, which prevents the binding of non-specific interactions with cells or body fluids. Thus the detection molecules can be directly linked or the enrichment and separation of target molecules or target cells from the functionalised surfaces can be simplified.

In an advantageous further improvement of the invention, the Biodetector features a pipe section, which meets at least one of the following requirements:
An inner space of pipe section communicates with the opening and/or the interior of the fluid removal section. Thus, the interior of the pipe section can also absorb an amount of fluid.
The pipe section is connected to the fluid removal section, preferably detachable. Due to the modular design, the components of biodetector can be easily replaced. In particular, the fluid removal section or pipe section can be easily replaced, which is advantageous for hygienic reasons. The connected parts preferably comprise the standardized connectors for medical applications.
The pipe section is designed in the form of a flexible hose. This allows the pipe section to be bent easily which is convenient for particular applications.
The pipe section has a larger internal diameter and/or outer diameter than the fluid removal section. Thereby the pipe section and the fluid removal section can be easily inserted into each other, wherein the pipe section is preferably attached or pushed onto the fluid removal section and is held onto the fluid removal section solely by frictional or elastic forces. Complicated connection mechanisms can be eliminated.
The pipe section comprises an inner diameter of 0.25 to 3.5 mm, preferably 0.5 to 3.0 mm, more preferably 0.75 to 2.5 mm, even more preferably 1.0 mm to 2.0 mm. With this internal diameter, the ratio of the amount of fluid coming into contact with the functionalised surface to the amount of the fluid absorbed by the interior space is particularly advantageous.
The pipe section comprising at least one branch pipe. Through a branch pipe, the ratio of the amount of fluid coming into contact with the functionalised surface to the amount of the fluid absorbed by the interior space is increased.
The pipe section comprising at least an open branch pipe and/or at least one short-circuited branch pipe and/or at least one branching pipe. The open branch pipe has a dead end and is flowed through with little or no velocity. The short-circuited branch pipe is connected at both ends with a different pipe and is passed through at a higher velocity than the open branch pipe. Depending on the application, an open or short branch pipe may be useful. Of course, a branch pipe can again have one or more branches, so that an arbitrarily complex pipeline system can be generated.
The pipe section comprises at least three branches, which extend to different levels. Thereby the branch pipes are arranged in a particularly compact form.
The pipe section comprises at least one cross sectional change, preferably at least one cross-sectional extension and/or at least one cross-sectional reduction. In a cross-sectional change the flow conditions are changed, in particular the flow velocity of the fluid absorbed in the pipe section. Precisely here, it may be useful to provide a functionalised surface.
The functionalised surface is located on an inner wall of the pipe section. Thereby the interior of the pipe section can be used for the enrichment of the target molecules or target cells.
The pipe section is preferably made of a plastic, more preferably a polymer, and even more preferably polystyrene. Such materials exhibit advantageous properties for binding of detection molecules. Antibodies can be adsorbed in polystyrene in the form of detection molecules. There is a wide range of polymers for optimal linker chemistry.
The pipe section consists at least partially of a material which contains functional groups for covalent binding of the detection molecule, and/or which contains chemically or enzymatically fissile groups, to facilitate the quantitative recovery of bound target molecules, or target cells, and/or forms a matrix, which prevents the binding of non-specific interactions with cells or body fluids. Thus the detection molecules can be directly linked or the enrichment and separation of target molecules or target cells from the functionalised surfaces can be simplified.

In another advantageous refinement of the invention, the Biodetector features a storage device that meets at least one of the following requirements:
The storage device comprises a variable volume. Thereby the amount of fluid absorbed in Biodetector is finely adjusted.
The interior of the storage device communicates with the opening and/or with the interior of the fluid removal section and/or the interior of the pipe section. By increasing the storage volume, the entire interior of the biodetector can be filled with the fluid. By reducing the storage capacity, the interior of biodetector can be at least partially emptied again.
The storage device is connected to the pipe section, preferably detachable. The modular design of biodetector according to this embodiment facilitates the exchange of individual components for various applications.
The storage device is designed in the form of a syringe. The use of standardized components according to this embodiment reduces the manufacturing cost of the biodetector.
The functionalised surface is located on an inner wall of the storage device. Thereby the interior of the storage device can be used for the enrichment of the target molecules or target cells.
The storage device is at least partially made of a material, which contains functional groups for covalent binding of the detection molecule, and/or which contains chemically or enzymatically fissile groups to facilitate the quantitative recovery of bound target molecules, or target cells, and/or forms a matrix, which prevents the binding of non-specific interactions with cells or body fluids. Thereby the detection molecules can be linked directly to the functionalised surface of the storage device or the enrichment and separation of the target molecules or the target cells can be simplified.

In yet another advantageous refinement of the invention, the Biodetector features a detection device that meets at least one of the following requirements:
The detection device is designed in the form of a functionalised chip. This allows already within the biosensor a more precise analysis, in particular a computer-aided detection and/or identification of the enriched target molecules or target cells to be performed, for example, with regard to the amount of the enriched target molecules or target cells per volume of fluid absorbed and so forth. The detection device preferably has an interface for data transmission.
The detection device is used for optical, preferably microscopically assisted detection and/or identification of the target molecules, or target cells. In this case it is preferable that at least a part of the detection device, which is provided with a functionalised surface, is designed to be transparent or translucent. This part of the detection device can be installed preferably in an optical detection device, for example a microscope, in such a way, that the target molecules or the target cells, which are optionally arranged in the detection device on the functionalised surface, can be detected by the optical detection device.
The detection device is used for computer-aided detection and/or identification of the target molecules, or target cells. Thereby the costs of detection and/or identification of the target molecules and target cells can be reduced (e.g., number of target molecules or cells per volume of fluid) significantly.
The detecting device is integrated into the fluid removal section and/or in the pipe section and/or in the storage device. Preferably, the detecting device is integrated into the pipe section. Thereby the detection device can be easily coupled with standard components such as cannula and syringe.
The interior space of detection device communicates with the opening and/or the interior of the fluid removal section and/or with the interior of the pipe section and/or with the interior space of the storage device. Thereby the detection device can be filled directly and without decanting the fluid removed from the body.

- The detection device is connected to the fluid removal section and/or to the pipe section and/or to the storage device, preferably detachable. The modular design of biodetector according to this embodiment facilitates the exchange of individual components for various applications. Further, the detection device for the detection and/or identification of the target molecules, or target cells can be easily detached from the other components of the biodetector.
The functionalised surface is located on an inner wall of the detection device. Thereby, the target molecules, or target cells in the detection device can be simultaneously enriched and analysed. A separation of the target molecules or the detection molecules from the target cells for the purposes of the analysis is therefore not required.
The detection device is at least partially made of a material, which contains functional groups for covalent binding of the detection molecule, and/or that contains chemically or enzymatically fissile groups, to facilitate the quantitative recovery of bound target molecules, or target cells, and/or forms a matrix, which prevents the binding of non-specific interactions with cells or body fluids. Thereby the detection molecules are linked directly to the functionalised surface of the detection device or the enrichment and separation of target molecules or target cells can be simplified.

In yet another further advantageous refinement of the invention, the Biodetector features a secondary layer, which meets at least one of the following requirements:
The secondary layer is designed in the form of polymer layer, preferably a hydrogel. The polymer layer facilitates the linking of the indicator molecules. Through a hydrogel, an enrichment of target molecules or target cells of functionalised surface can be increased considerably.
The secondary layer contains functional groups for covalent bonding of the detection molecules.
The secondary layer comprises chemically or enzymatically fissile groups, to facilitate the quantitative removal of bound target molecules, or target cells.
The secondary layer forms a matrix, which prevents the bonding of non-specific cells or interactions with body fluids.
Detection molecules are covalently bonded to the secondary layer.

To improve the efficiency of the Biodetector and enlarging of the functionalised surface, the functionalised surface itself and/or the substrate on which the functionalised surface is provided can have a patterning. The patterning may comprise projections and/or depressions, which can be designed, for example cylindrical, spherical segment, conical or frustoconical, pyramidal or truncated pyramidal shape. Possible, however, is also a patterning in the form of grooves, in particular the longitudinal and/or transverse grooves. Preferably, at least one of the inner walls of the fluid removal portion and/or the line section and/or the memory device and/or of the detection device features such a structure.

Further advantageous embodiment of the invention results from combination of the features or partial features mentioned in the claims.

### BRIEF DESCRIPTION OF THE DRAWINGS

FIG. 1 shows a schematic view of a first exemplary embodiment of the invention, wherein the biodetector is designed in the form of syringe with cannula.

FIG. 2 shows a schematic view of a second exemplary embodiment of the invention, wherein the biodetector is designed in the form of syringe with needle and interconnected hose.

FIG. 2 shows a schematic view of a third exemplary embodiment of the invention, wherein the biodetector is designed in the form of syringe with needle and interconnected hose with functionalised chip.

### DETAILED DESCRIPTION OF THE PREFERRED EXEMPLARY EMBODIMENT

The Biodetector 1 according to the invention and its individual components are described in detail below with reference to the accompanying drawings.

### Biodetector

The Biodetector 1 according to the invention is used for the enrichment and isolation of molecules or cells from the human body. For this purpose the Biodetector 1 is configured such that a fluid, in particular blood, is removed from the human body and can be absorbed in an interior space 20, 30, 40, 50 of the Biodetector 1. According to the invention, a functionalised surface 21, 31, 41, is at least partially filled with detection molecules, which faces the interior space 20, 30, 40, 50 of the Biodetector 1. This means that fluid absorbed in the interior 20, 30, 40, 50 of the biodetector can directly come into contact with the detection molecules, in order to be isolated or enriched. Preferably various functionalised surfaces 21, 31, 41, 51, if necessary for different target molecules, or target cells, in different sections 2, 3, 4, 5 of the Biodetector 1 are provided.

### Functionalised Surface

The preferred detection molecules or receptors or ligands on the functionalised surface, for example, monoclonal antibodies are of murine origin, chimeric antibodies, humanized antibodies, antibody fragments or amino acid structures and amino acid sequences, nucleic acid structures or nucleic acid sequences, or carbohydrate structures or synthetic structures having specific affinity for cell surfaces or molecules. Detection molecules can be of non-human origin, in particular of animal origin. Of course, the functionalised surface can be filled with various detection molecules.

### Binding of the Detection Molecules

Depending on the base material of the respective section 2, 3, 4, 5 of the Biodetector 1 the detection molecules can directly or indirectly be linked (via one or more intermediate layers) to the functionalised surface 21, 31, 41, 51 (for the sake of simplicity no distinction is made between a “functionalised surface”, which already has the detection molecules “and a ” to be functionalised surface”, which is functionalised by binding of the detection molecules). The functionalised surface 21, 31, 41, 51 is located either in the area of the fluid-removal section 2 and/or of the pipe section 3 and/or the storage device 4 and/or the detection device 5.

For the indirect binding of the detection molecules covalently, a secondary layer consisting of a functional, preferably biocompatible polymer, for example hydrogel, can be applied in the sub-surface through wet chemical processes. This functional polymer can comprise organic functional groups, which are able to covalently bind to ligands or receptors. The type of the functional groups of the secondary layer depends on the molecular properties of the specific ligands and receptors. Additionally, in this polymer there can be chemically or enzymatically fissile groups in order to facilitate the quantitative removal of bound target molecules, or target cells. If necessary, the secondary layer forms a matrix, which prevents the bonding of non-specific cells or interactions with cells or body fluids.

For direct binding of the detection molecules to one of the sections 2, 3, 4, 5 of the Biodetector 1 the substrate should (at least the part to be provided on which the surface is functionalised) include at least one of the above characteristics of the secondary layer. For enlarging of the functionalised surface, the functionalised surface itself and/or the substrate on which the surface is functionalised to provide, have a patterning.

In one example, monoclonal antibodies are of murine origin as detection molecules using Protein G covalently linked and oriented to the inner wall of the fluid removal section 2 and/or of the pipe section 3 and/or the storage device 4 and/or the detection device 5.

### First Exemplary Embodiment

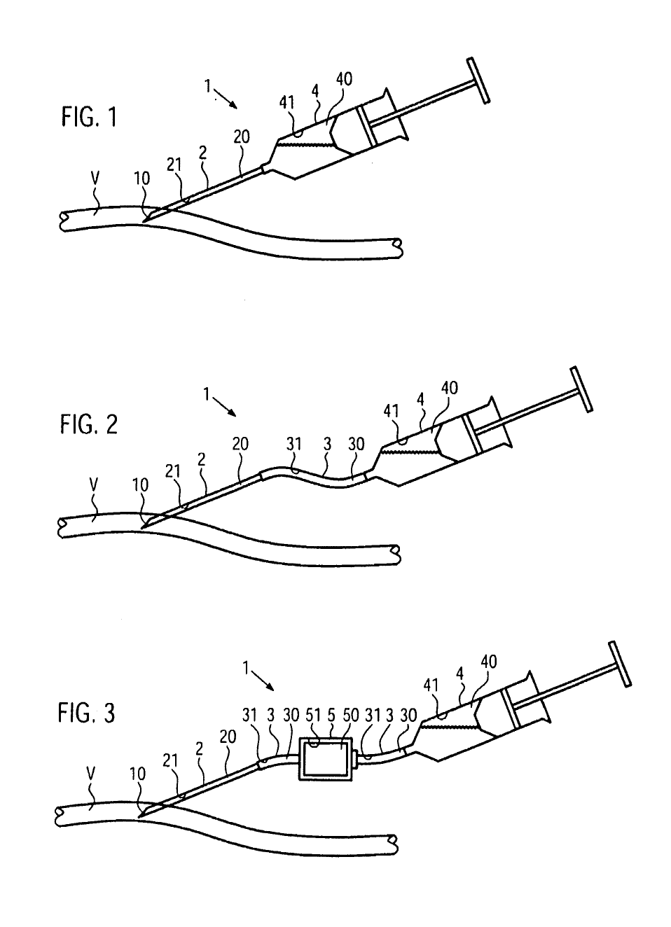

In the first embodiment example, which is shown schematically in FIG. 1, the according to invention Biodetector 1 comprises a fluid removal section 2, which is detachable connected to a storage device 4. The functionalised surface is located on an inner wall 21 of the fluid removal section 2 and/or on the inner wall 41 of the storage device 4

Fluid removal section 2 is formed as a hollow needle or cannula from a biocompatible material, particularly stainless steel, titanium, glass fibre, a biocompatible plastic or a combination of these substances. The appearance or the shape of the fluid removal section 2 can be variable and is dependent on the embodiment. Fluid removal section 2 is intended to cut through the skin and the tissue of a human body or to pierce through. Via an opening 10 at the front end, a fluid from the human body in the interior space 20, 40 can reach the Biodetector 1.

The storage device 4 is a syringe and is connected detachable to the fluid removal section 2, such that the inner space 40 of the storage device 4 communicates with the opening 10 and with the interior 20 of the fluid removal section 2. The interior 40 of the storage device 4 has a variable volume. By enlarging the interior space 40 of the storage device 4 via the communicating inner spaces 20, 40, a negative pressure is generated so that a fluid from the human body passes through an opening 10 into the interior 20, 40 of the Biodetector 1. By reducing the inner space 40 of the storage device 4, the fluid can be again expelled from the interior 20, 40 of the Biodetector 1.

### Second Exemplary Embodiment

The second exemplary embodiment, which is shown schematically in FIG. 2, corresponds largely to the first exemplary embodiment. Differing from the first exemplary embodiment, a line section 3 between the fluid removal section 2 and the storage device 4 is arranged. The functionalised surface is located on the inner wall 21 of the fluid removal section 2 and/or on the inner wall 31 of the pipe section 3 and/or on the inner wall 41 of the storage device 4

The pipe section 3 is formed as a flexible polymer tube, in particular as polystyrene tube and detachable connected to the fluid removal section 2 and the storage device 4 so that an inner space 30 of the pipe section 3, with the opening 10 and communicates with the interior 20, 40 of the fluid removal portion 2 and the storage device 4. Deviating from the illustration in FIG. 2 the pipe section 3 can have a cross-sectional variation and/or at least one branch pipe with open, short circuited and/or branching pipes.

### Third Exemplary Embodiment

The third exemplary embodiment, which is shown schematically in FIG. 3, largely corresponds to the second exemplary embodiment. Deviating from the second exemplary embodiment, a detection device 5 in the pipe section 3 is integrated such that an inner space 50 of the detection device 5 communicates with the opening 10 and with the interior spaces 20, 30, 40 of the fluid removal section 2, of the pipe section 3 and the storage device 4. The functionalised surface is located on the inner wall 21 of the fluid removal section 2 and/or on the inner wall 31 of the pipe section 3 and/or on the inner wall 41 of the storage device 4 and/or on the inner wall 51 of the detection device 5. The detection device 5 is designed in the form of functionalised chip for optical or computer-aided detection and identification of the target molecules and target cells.

### Application of Biodetectors

Fluid removal section 2 is intended to be introduced through the skin into a human body so that the opening 10 within the body and the other end of the fluid removal section 2 are located outside the body. Preferably, the opening 10 is located within a blood vessel V.

By drawing the syringe 4 and the associated suction effect fluid is removed from the human body, and conducted via the opening 10 into the interior space 20, 30, 40, 50 of the Biodetector 1, so that the fluid can come into contact with the functionalised surface 21, 31, 41, 51, and the fluid contained in the target molecules and target cells can reach the functionalised surface 21, 31, 41, 51. In FIGS. 1, 2 and 3 the inner space 40 of the storage device 4 is each partly filled with the fluid removed from the human body.

### Application Examples

The Biodetector 1 according to the invention is suitable for the recovery of rare cells from body fluids, especially from the bloodstream. This includes the following application examples:
Removal of embryonic cells from the maternal blood circulation with e.g. specific antibody fragments (F (ab) fragments), and murine monoclonal antibodies (IgG), which are typical cell surface proteins of embryonic cells, such as HLA-G can be recognized.
Removal of disseminated tumour cells, particularly of hematogenous metastatic tumours e.g. with the humanized anti-EpCAM antibody, which is typical for many cancer cells, which for many cancer cells is typical cell surface protein EpCAM.

The Biodetector 1 according to the invention is also suitable for the recovery of rare molecules from the bloodstream, particularly tumour markers or biomarkers.

The Biodetector 1 according to the invention is further suitable also for the elimination of drugs from the bloodstream. This includes the following applications examples:
Elimination of radioactive tracers for diagnosis and therapy.
Elimination of magnetic beads or super paramagnetic particles coupled with drugs.
Elimination of toxins

These application examples are merely illustrative and are not offered as exhaustive.

### Advantages of the Invention

According to the invention, a certain amount of body fluid, particularly blood, can be removed from the human body and absorbed in the interior of Biodetector 1 and analysed. The blood coming into contact with the functionalised surface is not fed back into the body of the subject and is no longer part of the blood circulation. Thereby, the use of human detection molecules is no longer required and can be omitted in all the biocompatibility tests. For example, detection molecules of animal origin, in particular monoclonal antibodies of murine origin can be used which by means of Protein G oriented are linked. The blood can be taken slowly through the cannula 2, so no problems with the negative effects of shear forces that can prevent blood flowing in the cell binding, are expected. Further, the blood can be pushed many times after removal through the needle 2, so that the removed quantity of blood is used twice for the binding of the target molecules and target cells. Based on the invention, using the modular structure of the Biodetector 1, the functionalised surface is enlarged as desired. Appropriate measures for enlarging the functionalised surface can be taken, so that with a relatively small volume of fluid more cells from the body fluid can come into contact with the functionalised surface. For this purpose, branch pipes, fine ramifications (see human lung) and/or cross-sectional changes can be provided. Inside the Biodetector 1 the functionalised surface and the enriched target molecules and target cells are well protected, whereby an abrasion can be prevented when introducing the Biodetector 1 in the body as well as by subsequent removal. This provides further advantages for the subsequent handling. The detected or isolated cells and molecules can be relatively easily washed and lysed. In contrast to alternative methods (Cell Search), the body fluid must therefore not be sent to appropriate laboratories to isolate cells and molecules. Procedures and equipment are not required for use of magnetic beads. The used devices or utensils can be handled easily and are mobile and can be used anywhere.

1. Biodetector with comprising:
a functionalised surface for isolation of molecules or cells from a human body, wherein the Biodetector is configured to remove a fluid from the human body, and to absorb it into an inner space of the Biodetector, wherein the functionalised surface is oriented to the inner space of the Biodetector.

2. Biodetector according to claim 1, wherein the functionalised surface is at least partially occupied with detection molecules.

3. Biodetector according to claim 2, wherein the detection molecule includes antibodies, selected from a group consisting of monoclonal antibodies, chimeric antibodies, humanized antibodies, antibody fragments or amino acid structures and amino acid sequences, nucleic acid structures and nucleic acid sequences, carbohydrate structures, and synthetic structures.

4. Biodetector according to claim 2, wherein the detection molecules are of non-human origin.

5. Biodetector according to claim 1, wherein the detection molecules are connected and oriented to the functionalised surface.

6. Biodetector according to claim 1, wherein the detection molecules are covalently attached to the functionalised surface.

7. Biodetector according to claim 1, wherein the Biodetector has a fluid removable section, which satisfies at least one of the following requirements:
a. The fluid removal section comprises an opening through which the fluid passes into the inner space of the Biodetector;
b. An inner space/interior of the fluid removal section communicates with the opening;
c. The opening is formed at one end of the fluid removal section;
d. Fluid The fluid removal section is formed from a biocompatible material;
e. The fluid removal section is made of plastic, metal or glass, or a combination of these substances;
f. The fluid removal section is designed as a cannula;
g. The fluid removal section comprises an outer diameter of 0.25 to 3.5 mm;
h. The opening comprises a diameter of 0.2 to 3.0 mm;
i. The functionalised surface is located on an inner wall of the fluid removal section; and
j. Fluid removal section comprises a material, which contains functional groups for covalent bonding of the detection molecules, and/or contains chemically or enzymatically fissile groups, to facilitate quantitative removal of bound target molecules, or target cells, and/or forms a matrix which prevents bonding of non-specific cells or interactions with cells or body fluids.

8. Biodetector according to claim 7, wherein that the Biodetector has a pipe section, which satisfies at least one of the following requirements:
a. An inner space of the pipe section communicates with an opening and/or an interior of a fluid removal section;
b. The pipe section is connected to the fluid removal section;
c. The pipe section is configured as a flexible hose;
d. The pipe section has a larger internal diameter and/or outer diameter than the fluid removal section;
e. The pipe section comprises an inner diameter of 0.25 to 3.5 mm;
f. The pipe section comprises at least one branch pipe;
g. The pipe section comprises at least an open branch pipe and/or at least one short-circuited branch pipe and/or at least one branching pipe;
h. The pipe section comprises at least three branch pipes, which extend in different planes;
i. The pipe section comprises at least one change in cross section;
j. The functionalised surface is located on an inner wall of the pipe section;
k. The pipe section is made of plastic; and
l. The pipe section comprises a material, which contains functional groups for covalent binding of the detection molecule, and/or chemically or enzymatically fissile groups to facilitate quantitative recovery of bound target molecules, or target cells, and/or forms a matrix which prevents binding of non-specific interactions with cells or body fluids.

9. Biodetector according to claim 8, wherein the Biodetector has a storage device which satisfies at least one of the following requirements:
a. The storage device comprises a variable volume;
b. The interior of the storage device communicates with the opening and/or with the interior of the fluid removal section and/or the interior of the pipe section;
c. The storage device is connected to the pipe section and/or connected to the fluid removal section;
d. The storage device is configured as a syringe;
e. The functionalised surface is located on an inner wall of the storage device; and
f. The memory storage device comprises a material, which contains functional groups for covalent binding of the detection molecule, and/or which contains chemically or enzymatically fissile groups, to facilitate quantitative recovery of bound target molecules, or target cells, and/or forms a matrix which prevents binding of non-specific interactions with cells or body fluids.

10. Biodetector according to claim 9, wherein the Biodetector has a detection device which satisfies at least one of the following requirements:
a. The detection device is configured as a functionalised chip;
b. The detection device provides optical, detection and/or identification of target molecules, or target cells;
c. The detection device provides computer-aided detection and/or identification of the target molecules, or target cells;
d. The detection device is integrated into the fluid removal section and/or in the pipe section and/or the storage device;
e. The inner space of the detection device communicates with the opening and/or with the interior of the fluid removal section and/or the interior of the pipe section and/or with the interior of the storage device;
f. The detection device is connected to the fluid removal section and/or to the pipe section and/or to the storage device;
g. The functionalised surface is located on an inner wall of the detection device; and
h. The detection device comprises a material, which contains functional groups for covalent binding of the detection molecule, and/or that contains chemically or enzymatically fissile groups, to facilitate quantitative recovery of bound target molecules, or target cells, and/or forms a matrix which prevents binding of non-specific interactions with cells or body fluids.

11. Biodetector according to claim 10, wherein the Biodetector has a secondary layer which satisfies at least one of the following requirements:
a. The secondary layer is designed in the form of as a polymer layer;
b. The secondary layer contains functional groups for covalent bonding of the detection molecules;
c. The secondary layer comprises chemically or enzymatically fissile groups, to facilitate quantitative removal of bound target molecules, or target cells;
d. The secondary layer forms a matrix, which prevents bonding of non-specific cells or interactions with body fluids; and
e. At the secondary layer detection molecules are covalently bonded.

12. Biodetector according to claim 1, wherein the functionalised surface has a patterning, with projections and/or recesses, which are at least one of cylindrical, spherical segment, conical or frustoconical, pyramidal or truncated pyramidal shape, or ridges or furrows.
